(19) United States
(12) Patent Application Publication
(10) Pub. No.: US 2011/0152990 A1
(43) Pub. Date: Jun. 23, 2011

(54) MRI COMPATIBLE LEAD EMPLOYING MULTIPLE MINIATURE INDUCTORS

(75) Inventors: Ramez Shehada, La Mirada, CA (US); Xiaoyi Min, Thousand Oaks, CA (US); Jin Zhang, Northridge, CA (US); Benjamin Coppola, Woodland Hills, CA (US); Dave Anderson, Castaic, CA (US); Gene A. Bornzin, Simi Valley, CA (US); Phong D. Doan, Stevenson Ranch, CA (US)

(73) Assignee: PACESETTER, INC., Sylmar, CA (US)

(21) Appl. No.: 12/645,252

(22) Filed: Dec. 22, 2009

(51) Int. Cl. A61N 1/05; H03H 7/01

(52) U.S. Cl. 607/127; 607/122; 333/176

(57) ABSTRACT

An implantable medical lead is disclosed herein. The lead includes a first electrode and a first electrical circuit. The first electrode is near a distal portion of the lead. The first electrical circuit extends through the lead to the first electrode and includes at least one conductor and a first band stop filter coupled between the distal end of the conductor and the electrode. The first band stop filter includes a first group of inductors in parallel and a second group of inductors in parallel. The first group is in series with the second group. The first group of inductors may include a self resonant L. The first group of inductors may include a self resonant tank LC. The first group of inductors may include a miniature self resonant L or miniature self resonant tank LC. The first group of inductors may include an integrated circuit of L and C components.

### FIELD OF THE INVENTION

The present invention relates to implantable medical leads. More specifically, the present invention relates to implantable medical leads configured to result in reduced heating when subjected to MRI.

### BACKGROUND OF THE INVENTION

Existing implantable medical leads for use with implantable pulse generators, such as neurostimulators, pacemakers, or implantable cardioverter defibrillators (“ICD”), are prone to heating and induced current when placed in the strong magnetic (static, gradient and RF) fields of a magnetic resonance imaging (“MRI”) machine. The heating and induced current are the result of the lead acting like an antenna in the magnetic fields generated during a MRI. Heating and induced current in the lead may result in deterioration of stimulation thresholds or, in the context of a cardiac lead, even increase the risk of cardiac tissue damage and perforation.

Over fifty percent of patients with an implantable pulse generator and implanted lead require, or can benefit from, a MRI in the diagnosis or treatment of a medical condition. MRI modality allows for flow visualization, characterization of vulnerable plaque, non-invasive angiography, assessment of ischemia and tissue perfusion, and a host of other applications. The diagnosis and treatment options enhanced by MRI are only going to grow over time. For example, MRI has been proposed as a visualization mechanism for lead implantation procedures.

There is a need in the art for an implantable medical lead configured for improved MRI safety. There is also a need in the art for methods of manufacturing and using such a lead.

### BRIEF SUMMARY OF THE INVENTION

An implantable medical lead is disclosed herein. In one embodiment the lead includes a first electrode and a first electrical circuit. The first electrode is near a distal portion of the lead. The first electrical circuit extends through the lead to the first electrode and includes at least one conductor and a first band stop filter coupled between a distal end of the conductor and the electrode. The first band stop filter includes a first group of inductors in parallel and a second group of inductors in parallel. The first group is in series with the second group. The first group of inductors may include a self resonant L. The first group of inductors may include a self resonant tank LC. The first group of inductors may include a miniature self resonant L or miniature self resonant tank LC. The first group of inductors may include an integrated circuit of L and C components.

Another implantable medical lead is disclosed herein. In one embodiment the lead includes a first electrode and a first electrical circuit. The first electrode is near a distal portion of the lead. The first electrical circuit extends through the lead to the first electrode and includes at least one conductor and a first band stop filter coupled between a distal end of the conductor and the electrode. The first band stop filter includes a first group of inductors in series and a second group of inductors in series. The first group is in parallel with the second group.

While multiple embodiments are disclosed, still other embodiments of the present invention will become apparent to those skilled in the art from the following Detailed Description, which shows and describes illustrative embodiments of the invention. As will be realized, the invention is capable of modifications in various aspects, all without departing from the spirit and scope of the present invention. Accordingly, the drawings and detailed description are to be regarded as illustrative in nature and not restrictive.

### BRIEF DESCRIPTION OF THE DRAWINGS

FIG. 1 is an isometric view of an implantable medical lead and a pulse generator for connection thereto.

FIG. 2 is a longitudinal cross-section of the lead distal end.

FIGS. 3A-3B are side views of alternative embodiments of band stop filters.

FIGS. 4A-4D are transverse cross sections of the band stop filters as taken along section lines 4-4 in FIGS. 3A and 3B.

FIG. 5 is a graph comparing performance of various band stop filter configuration.

FIGS. 6A and 6B are diagrammatic side views of different embodiments of a band stop filter assembly for use with a circuit leading to a tip electrode.

FIG. 7A is a diagrammatic side view of an embodiment of a band stop filter assembly for use with a circuit leading to a ring electrode.

FIG. 7B is a transverse cross section of the band stop filter as taken along section line 7B-7B of FIG. 7A.

FIG. 8 is a diagrammatic depiction if a micro inductor circuit.

FIGS. 9A and 9B are plan views of other micro-inductor circuits.

FIGS. 10A and 10B are side views of the embodiment depicted in FIG. 9A of a flexible substrate not flexed and flexed, respectively.

FIG. 11 is a graph depicting peak impedances for the embodiment of FIG. 9B.

### DETAILED DESCRIPTION

Disclosed herein is an implantable medical lead 10 employing band stop filters (e.g., inductor groups) 160, 190 in the electrical circuits leading to the respective electrodes 75, 80 at the distal portion 45 of the lead 10. In one embodiment, a band stop filter 160, 190 uses multiple miniature inductors (e.g., self resonant L or self resonant tank LC) 200 in a combination of parallel and serial connections. Such a band stop filter 160, 190 may be packaged with or without a hermitical seal. Also, such a band stop filter 160, 190 may allow for the elimination of the use of a Ti sleeve in a lead and allow a band stop filter to fit in existing lead dimensions or smaller. More importantly, employing the band stop filters 160, 190 disclosed herein will reduce inductor heating by distributing the energy among the multiple inductors 200 and provide better reliability due to the inductors 200 being in parallel, as opposed to being in serial.

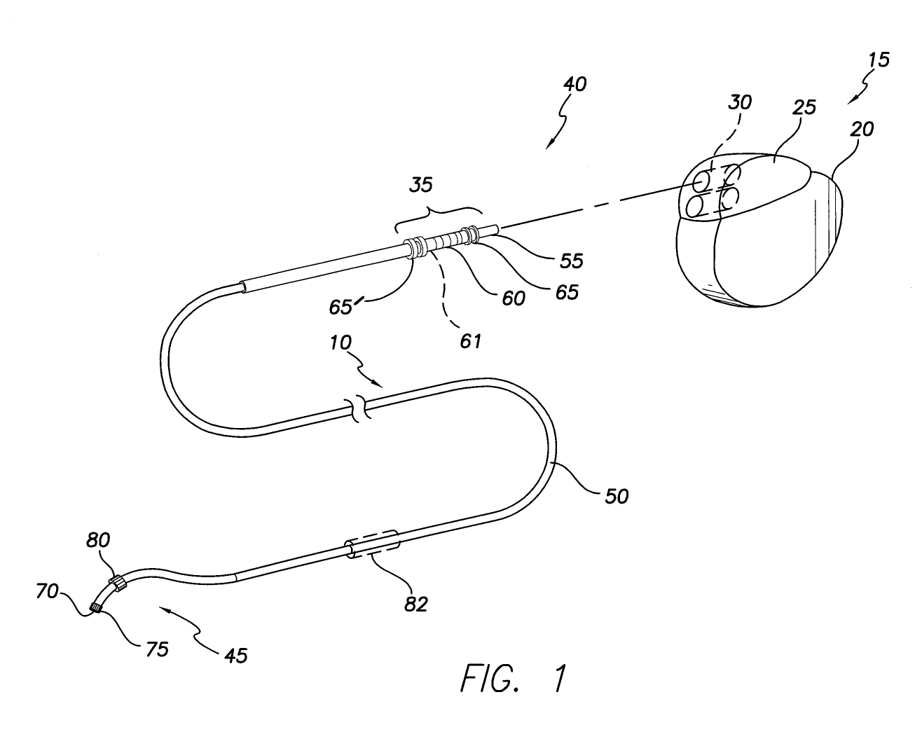

For a general discussion of an embodiment of a lead 10 employing the band stop filters (e.g., inductor groups) 160, 190, reference is made to FIG. 1, which is an isometric view of the implantable medical lead 10 and a pulse generator 15 for connection thereto. The pulse generator 15 may be a pacemaker, ICD or neurostimulator. As indicated in FIG. 1, the pulse generator 15 may include a can 20, which may house the electrical components of the pulse generator 15, and a header 25. The header 25 may be mounted on the can 20 and may be configured to receive a lead connector end 35 in a lead receiving receptacle 30. Although only a single lead is illustrated, it can be appreciated that multiple leads may be implemented. In particular, for example, for CRT treatments, there may be leads for both the right and left ventricle.

As shown in FIG. 1, in one embodiment, the lead 10 may include a proximal end 40, a distal end 45 and a tubular body 50 extending between the proximal and distal ends. The lead 10 may be configured for a variety of uses. For example, the lead 10 may be a RA lead, RV lead, LV Brady lead, RV Tachy lead, intrapericardial lead, etc.

As indicated in FIG. 1, the proximal end 40 may include a lead connector end 35 including a pin contact 55, a first ring contact 60, a second ring contact 61, which is optional, and sets of spaced-apart radially projecting seals 65. In some embodiments, the lead connector end 35 may include the same or different seals and may include a greater or lesser number of contacts. The lead connector end 35 may be received in a lead receiving receptacle 30 of the pulse generator 15 such that the seals 65 prevent the ingress of bodily fluids into the respective receptacle 30 and the contacts 55, 60, 61 electrically contact corresponding electrical terminals within the respective receptacle 30.

As illustrated in FIG. 1, in one embodiment, the lead distal end 45 may include a distal tip 70, a tip electrode 75 and a ring electrode 80. In some embodiments, the lead body 50 is configured to facilitate passive fixation and/or the lead distal end 45 includes features that facilitate passive fixation. In such embodiments, the tip electrode 75 may be in the form of a ring or domed cap and may form the distal tip 70 of the lead body 50.

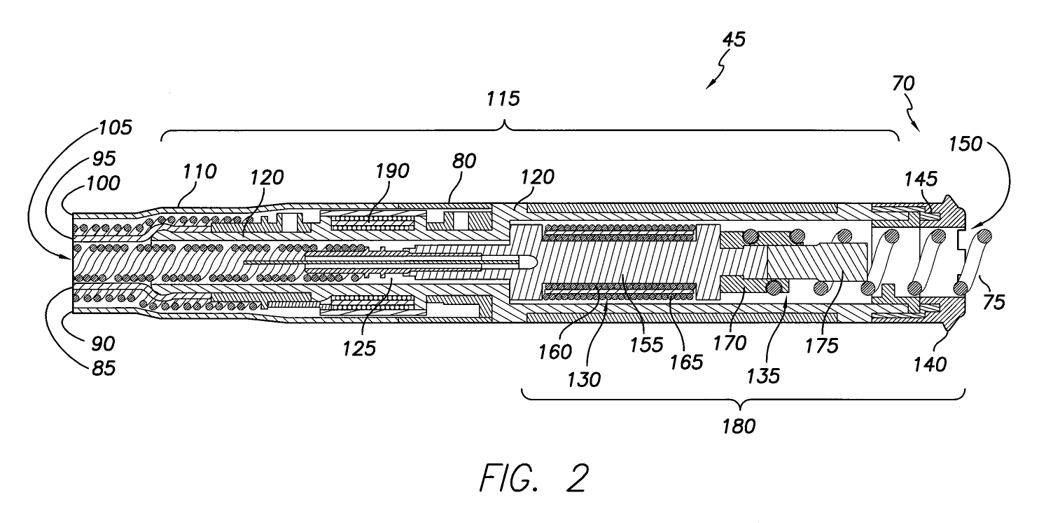

As shown in FIG. 2, which is a longitudinal cross-section of the lead distal end 45, in some embodiments, the tip electrode 75 may be in the form of a helical anchor 75 that is extendable from within the distal tip 70 for active fixation and serving as a tip electrode 75.

As shown in FIG. 1, in some embodiments, the distal end 45 may include a defibrillation coil 82 about the outer circumference of the lead body 50. The defibrillation coil 82 may be located proximal of the ring electrode 70.

The ring electrode 80 may extend about the outer circumference of the lead body 50, proximal of the distal tip 70. In other embodiments, the distal end 45 may include a greater or lesser number of electrodes 75, 80 in different or similar configurations.

As can be understood from FIGS. 1 and 2, in one embodiment, the tip electrode 75 may be in electrical communication with the pin contact 55 via a first electrical conductor 85, and the ring electrode 80 may be in electrical communication with the first ring contact 60 via a second electrical conductor 90. In some embodiments, the defibrillation coil 82 may be in electrical communication with the second ring contact 61 via a third electrical conductor. In yet other embodiments, other lead components (e.g., additional ring electrodes, various types of sensors, etc.) (not shown) mounted on the lead body distal region 45 or other locations on the lead body 50 may be in electrical communication with a third ring contact (not shown) similar to the second ring contact 61 via a fourth electrical conductor (not shown). Depending on the embodiment, any one or more of the conductors 85, 90 may be a multi-strand or multi-filar cable or a single solid wire conductor run singly or grouped, for example in a pair.

As shown in FIG. 2, in one embodiment, the lead body 50 proximal of the ring electrode 80 may have a concentric layer configuration and may be formed at least in part by inner and outer helical coil conductors 85, 90, an inner tubing 95, and an outer tubing 100. The helical coil conductor 85, 90, the inner tubing 95 and the outer tubing 100 form concentric layers of the lead body 50. The inner helical coil conductor 85 forms the inner most layer of the lead body 50 and defines a central lumen 105 for receiving a stylet or guidewire therethrough. The inner helical coil conductor 85 is surrounded by the inner tubing 95 and forms the second most inner layer of the lead body 50. The outer helical coil conductor 90 surrounds the inner tubing 95 and forms the third most inner layer of the lead body 50. The outer tubing 100 surrounds the outer helical coil conductor 90 and forms the outer most layer of the lead body 50.

In one embodiment, the inner tubing 95 may be formed of an electrical insulation material such as, for example, ethylene tetrafluoroethylene (“ETFE”), polytetrafluoroethylene (“PTFE”), silicone rubber, silicone rubber polyurethane copolymer (“SPC”), or etc. The inner tubing 95 may serve to electrically isolate the inner conductor 85 from the outer conductor 90. The outer tubing 100 may be formed of a biocompatible electrical insulation material such as, for example, silicone rubber, silicone rubber—polyurethane—copolymer (“SPC”), polyurethane, gore, or etc. The outer tubing 100 may serve as the jacket 100 of the lead body 50, defining the outer circumferential surface 110 of the lead body 50.

As illustrated in FIG. 2, in one embodiment, the lead body 50 in the vicinity of the ring electrode 80 transitions from the above-described concentric layer configuration to a header assembly 115. For example, in one embodiment, the outer tubing 100 terminates at a proximal edge of the ring electrode 80, the outer conductor 90 mechanically and electrically couples to a proximal end of the ring electrode 80, the inner tubing 95 is sandwiched between the interior of the outer conductor 90 and an exterior of a proximal end portion of a body 120 of the header assembly 115, and the inner conductor 85 extends distally past the ring electrode 80 to electrically and mechanically couple to components of the header assembly 115 as discussed below.

As depicted in FIG. 2, in one embodiment, the header assembly 115 may include the body 120, a coupler 125, a band stop filter assembly 130, and a helix assembly 135. The header body 120 may be a tube forming the outer circumferential surface of the header assembly 115 and enclosing the components of the assembly 115. The header body 120 may have a soft atraumatic distal tip 140 with a radiopaque marker 145 to facilitate the soft atraumatic distal tip 140 being visualized during fluoroscopy. The distal tip 140 may form the extreme distal end 70 of the lead 10 and includes a distal opening 150 through which the helical tip anchor 75 may be extended or retracted. The header body 120 may be formed of polyetheretherketone (“PEEK”), polyurethane, or etc., the soft distal tip 140 may be formed of silicone rubber, SPC, or etc., and the radiopaque marker 145 may be formed of platinum, platinum-iridium alloy, tungsten, tantalum, or etc.

As indicated in FIG. 2, in one embodiment, the band stop filter assembly 130 may include a bobbin 155, a band stop filter 160 and a shrink tube 165. The bobbin 155 may include a proximal portion that receives the coupler 125 such that the coupler 125 and bobbin 155 are mechanically coupled to each other. The bobbin 155 may also include a barrel portion about which the band stop filter 160 is located and a distal portion coupled to the helix assembly 135. The bobbin 155 may be formed of an electrical insulation material such as PEEK, polyurethane, or etc.

As illustrated in FIG. 2, the shrink tube 165 may extend about the band stop filter 160 to generally enclose the band stop filter 160 within the boundaries of the bobbin 155 and the shrink tube 165. The shrink tube 165 may act as a barrier between the band stop filter 160 and the inner circumferential surface of the header body 120. Also, the shrink tube 165 may be used to form at least part of a hermitic seal about the band stop filter 160. The shrink tube 165 may be formed of fluorinated ethylene propylene (“FEP”), polyester, or etc.

As shown in FIG. 2, a distal portion of the coupler 125 may be received in the proximal portion of the bobbin 155 such that the coupler 125 and bobbin 155 are mechanically coupled to each other. A proximal portion of the coupler 125 may be received in the lumen 105 of the inner coil conductor 85 at the extreme distal end of the inner coil conductor 85, the inner coil conductor 85 and the coupler 125 being mechanically and electrically coupled to each other. The coupler 125 may be formed of MP35N, platinum, platinum iridium alloy, stainless steel, etc.

As indicated in FIG. 2, the helix assembly 135 may include a base 170, the helical anchor electrode 75, and a steroid plug 175. The base 170 forms the proximal portion of the assembly 135. The helical anchor electrode 75 forms the distal portion of the assembly 135. The steroid plug 175 may be located within the volume defined by the helical coils of the helical anchor electrode 75. The base 170 and the helical anchor electrode 75 are mechanically and electrically coupled together. The distal portion of the bobbin 155 may be received in the helix base 170 such that the bobbin 155 and the helix base 170 are mechanically coupled to each other. The base 170 of the helix assembly 135 may be formed of platinum, platinum-iridium alloy, MP35N, stainless steel, or etc. The helical anchor electrode 75 may be formed of platinum, platinum-iridium ally, MP35N, stainless steel, or etc.

As can be understood from FIG. 2 and the preceding discussion, the coupler 125, band stop filter assembly 130, and helix assembly 135 are mechanically coupled together such that these elements 125, 130, 135 of the header assembly 115 do not displace relative to each other. Instead these elements 125, 130, 135 of the header assembly 115 are capable of displacing as a unit relative to, and within, the body 120 via the pin contact 55, which is rotatable relative to the rest of the lead connector end 35 and is mechanically and electrically coupled to the proximal end of the inner coil 85, the inner coil 85 being rotatable relative to the rest of the lead body 50. In other words, these elements 125, 130, 135 of the header assembly 115 form an electrode-band stop filter assembly 180, which can be caused to displace relative to, and within, the header assembly body 120 when a pin contact 55 and the inner coil 85 are caused to rotate within the lead connector end 35 and the lead body 50, respectively. Specifically, the pin contact 55 is rotated relative to the lead connector end 35, which causes the inner coil 85 to rotate relative to the lead body 50, which in turn causes the electrode-band stop filter assembly 180 to rotate within the header assembly of the lead distal end. Thus, rotation of the electrode-band stop filter assembly 180 in a first direction via rotation of the pin contact 55 in the first direction causes the electrode-band stop filter assembly 180 to displace distally, and rotation of the electrode-band stop filter assembly 180 in a second direction opposite the first direction via rotation of the pin contact 55 in the second direction causes the electrode-band stop filter assembly 180 to retract into the header assembly body 120. Thus, causing the electrode-band stop filter assembly 180 to rotate within the body 120 in a first direction causes the helical anchor electrode 75 to emanate from the tip opening 150 for screwing into tissue at the implant site. Conversely, causing the electrode-band stop filter assembly 180 to rotate within the body 120 in a second direction causes the helical anchor electrode 75 to retract into the tip opening 150 to unscrew the anchor 75 from the tissue at the implant site.

As already mentioned and indicated in FIG. 2, the band stop filter 160 may be positioned about the barrel portion of the bobbin 155. A proximal end of the band stop filter 160 may extend through the proximal portion of the bobbin 155 to electrically couple with the coupler 125, and a distal end of the band stop filter 160 may extend through the distal portion of the bobbin 155 to electrically couple to the helix base 170. Thus, in one embodiment, the band stop filter 160 is in electrical communication with both the inner coil conductor 85, via the coupler 125, and the helical anchor electrode 75, via the helix base 170. Therefore, the band stop filter 160 acts as an electrical pathway through the electrically insulating bobbin 155 between the coupler 125 and the helix base 170. In one embodiment, all electricity destined for the helical anchor electrode 75 from the inner coil conductor 85 passes through the band stop filter 160 such that the inner coil conductor 85 and the electrode 75 both benefit from the presence of the band stop filter 160, the band stop filter 160 acting as self resonant lumped inductor 160 when the lead 10 is present in a magnetic field of a MRI.

As the helix base 170 may be formed of a mass of metal, the helix base 170 may serve as a relatively large heat sink for the band stop filter 160, which is physically connected to the helix base 170. Similarly, as the coupler 125 may be formed of a mass of metal, the coupler 125 may serve as a relatively large heat sink for the band stop filter 160, which is physically connected to the coupler 125.

Some lead embodiments may have both a tip band stop filter 160 and a ring band stop filter 190. In such embodiments, the ring band stop filter (e.g., ring inductor group) 190 is part of the electrical circuit extending between the ring electrode 80 and the outer conductor 90 and the tip band stop filter 160 is part of the electrical circuit between the tip electrode 75 and the inner conductor 85. In such an embodiment, decoupling or isolating of the tip band stop filter 160 from the ring band stop filter 190 may be implemented as one or more magnetic shielding layers (“shield”) or a non-magnetic, electrically conductive material are located between the band stop filters 160, 190. In other embodiments, shields may not be located between the band stop filters 160, 190 and the two band stop filters 160, 190 may not be magnetically decoupled.

Additionally, in some embodiments, the tip band stop filter 160 may have a self-resonant frequency (SRF) that is different from the SRF of the ring band stop filter 190. For example, one of the band stop filters 160, 190 may be tuned for a frequency of 64 MHz and the other of the band stop filters may be tuned for a frequency of 128 MHz. Alternatively, in some embodiments, the tip band stop filter 160 may have a SRF that is the same as the SRF of the ring band stop filter 190. For example, both of the band stop filters 160, 190 may be tuned for a frequency of 64 MHz or 128 MHz.

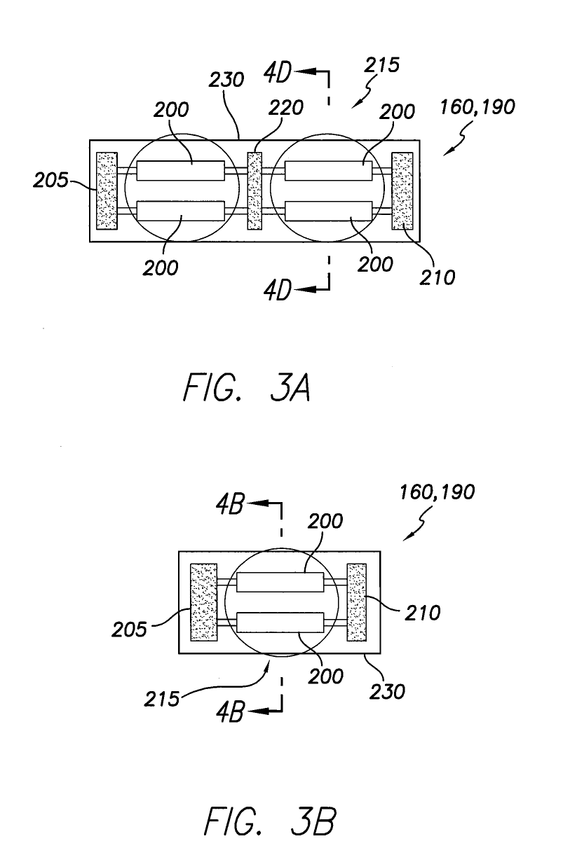

For a discussion of some various configurations of the band stop filters 160, 190, reference is first made to FIGS. 3A-3B, which are side views of alternative embodiments of band stop filters 160, 190. As shown in FIGS. 3A and 3B, the band stop filters 160, 190 are formed of multiple miniature inductors 200 that are electrically coupled together in parallel via a common proximal electrical contact 205 and a common distal electrical contact. As can be understood from FIGS. 1, 2, 3A and 3B, when a band stop filter 160, 190 is installed in the lead 10, the common proximal electrical contact 205 is electrically coupled to the electrical circuit leading from the band stop filter 160, 190 to the corresponding electrical contact of the lead connector end 35. Similarly, the common distal electrical contact 210 is electrically coupled to the electrical circuit leading from the band stop filter 160, 190 to the corresponding electrode 75, 80 at the lead distal end 45.

As shown in FIG. 3B, in some embodiments, a band stop filter 160, 190 may be a single group 215 of miniature inductors 200 wired in parallel, but not in series. As indicated in FIG. 3A, in other embodiments, a band stop filter 160, 190 may be multiple groups 215, 225 of miniature inductors 200 that are wired both in parallel and in series, a first group 215 of parallel wired miniature inductors 200 being wired in series via a common intermediate electrical contact 220 to a second group 225 of parallel wired miniature inductors 200. While two groups 215, 225 of parallel wired miniature inductors 200 wired in series are shown in FIG. 3A, in other embodiments, three, four or more groups of parallel wired miniature inductors 200 may be wired in series via the use of two, three or more common intermediate electrical contacts 220, such a contact 220 being located between each set of adjacent groups 215, 225 of parallel wired miniature inductors.

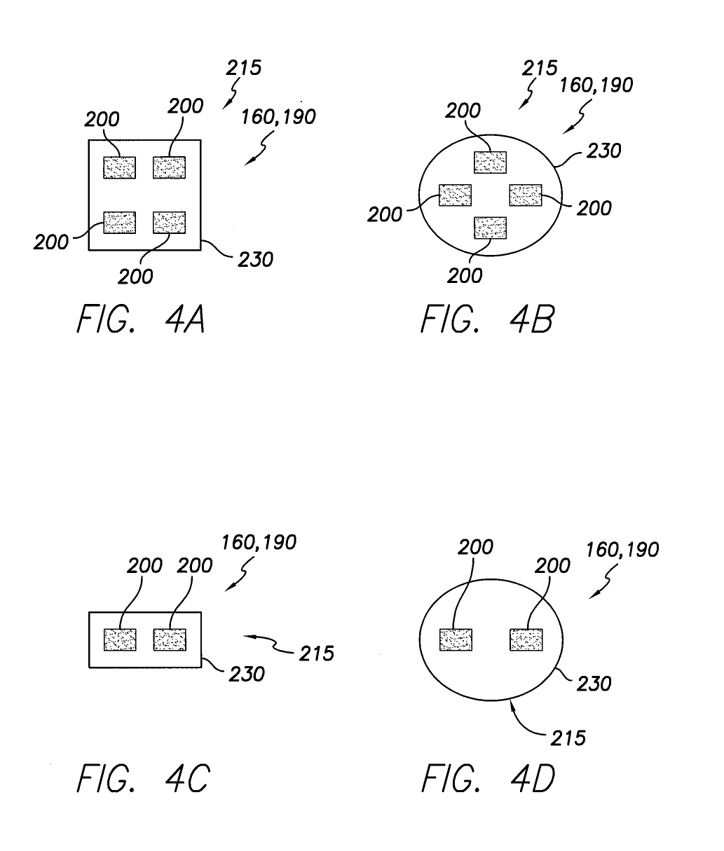

As can be understood from FIGS. 4A-4D, which are transverse cross sections of the band stop filters 160, 190 as taken along section lines 4-4 in FIGS. 3A and 3B, in some embodiments, groups 215 of parallel wired miniature inductors 200 may have two or four miniature inductors 200 wired in parallel. In other embodiments, the groups 215 of parallel wired miniature inductors 200 may have three, five, six, seven, eight, or more miniature inductors 200 wired in parallel.

In some embodiments, the miniature inductors 200 are the same or similar to those made by MediGuide, Ltd., MATAM—Merkaz Taasiot Mada, HAIFA 31053, ISRAEL. In one embodiment, the MediGuide micro-inductors may have dimensions of approximately 0.287-mm outer diameter and approximately 1-mm in length. In other words, such miniature inductors 200 may be as small as 270 micron in width by 1000 micron in length. With miniature inductors 200 of such a small size, a 6 Fr or 7 Fr lead may hold at least four or more such miniature inductors.

Such miniature inductors 200 may be made of 10 micron copper wires and with 100-400 turns and a non-ferrite core, inductance being in the range of approximately 3-6 uH. In embodiments of miniature inductors employing copper wires, the band stop filters 160, 190 may employ a hermetic seal 230, as shown in FIGS. 3A-4D. A hermetic seal 230 may not be needed if the miniature inductors 200 and the rest of the components of the band stop filters 160, 190 are made of biocompatible materials. For example, instead of copper wires being used to form the miniature inductors 200, DFT wires with 28%-50% Ag can be used and coated with ETFE.

In some embodiments, integrated circuits of inductive and capacitive components form the miniature inductors 200 and/or an entire band stop filter 160, 190. Thus, such integrated circuit miniature inductors 200 may be used with or in place of some or all of the coil miniature inductors 200 described above. In one embodiment, the miniature inductors may be an integrated LC in RF on a ceramic substrate as manufactured by Anaren Ceramics, Inc.

In some embodiments, regardless of whether a band stop filter 160, 190 is formed of a single group 215 of miniature inductors 200 wired in parallel (see FIG. 3B) or multiple series wired groups 215, 225 of miniature inductors 200 wired in parallel (see FIG. 3A), the electrical performance of total outcome for the band stop filter 160, 190 is generally equivalent to a single band stop filter (e.g., a self resonant inductor (L) or tank inductor/capacitor (LC)). However, unlike a single band stop filter, the above described band stop filter 160, 190 advantageously provides multiple connection points and reduced component heating. By providing multiple electrical connection points in parallel, if any one of the electrical connection points fails, the circuitry continues to work at even better electrical performance. By providing multiple inductors 200, the energy is distributed among the multiple inductors 200 so component heating is reduced. Depending on the bio-compatibility of the materials forming the components of the band stop filters 160, 190, the band stop filters 160, 190 may be packaged with or without hermetical seal for bio-compatibility.

In one embodiment, as can be understood from FIGS. 3A, 4C and 4D, two miniature inductors 200 wired in parallel may be in the first group 215 of inductors 200, and a two miniature inductors 200 wired in parallel may be in the second group 225 of inductors 200, the first and second groups 215, 225 having the same configuration and connected in serial to form a band stop filter 160, 190. In another embodiment, as can be understood from FIGS. 3A, 4A and 4B, four miniature inductors 200 wired in parallel may be in the first group 215 of inductors 200, and a four miniature inductors 200 wired in parallel may be in the second group 225 of inductors 200, the first and second groups 215, 225 having the same configuration and connected in serial to form a band stop filter 160, 190. Such parallel and series combinations of miniature inductors 200 may be employed to achieve the same impedance at frequency response as a single inductor while achieving circuit redundancy and reduced component heating.

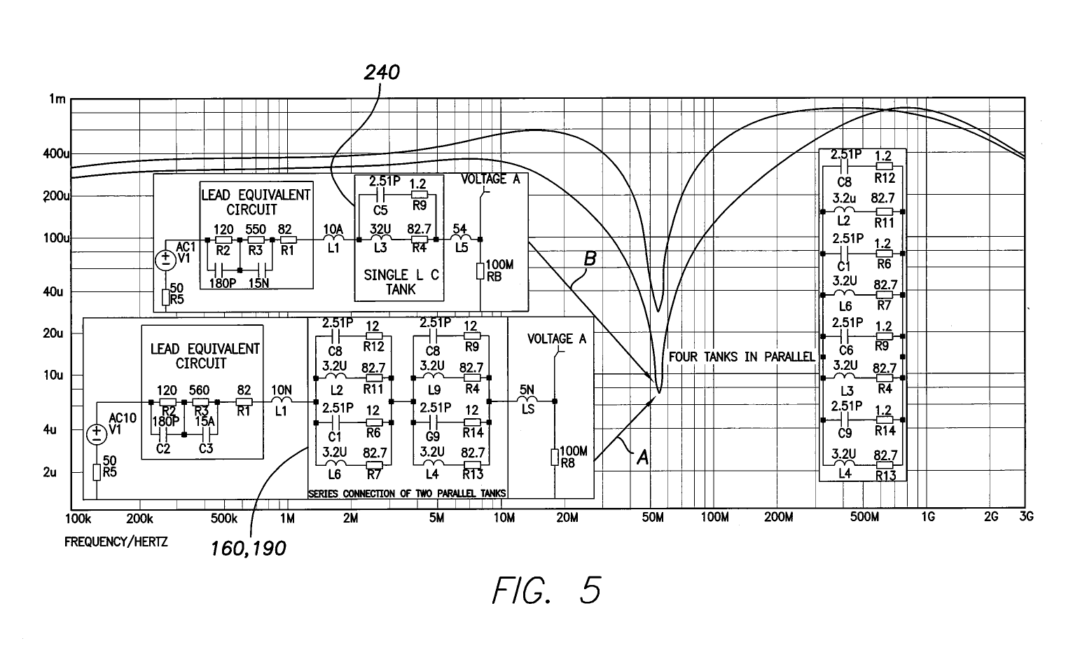

The advantages of the combination parallel and series wiring arrangement of the miniature inductors 200 can be understood from TABLE 1 (provided immediately below) and the graph depicted in FIG. 5. For example, band stop filter 160, 190 employed inductors having 3 mil 75 percent Ag DFT wire wound at 90 turns on a tip bobbin were tested in a circuit simulation. The band stop filter 160, 190 was configured as can be understood from FIGS. 3A, 4C and 4D (i.e., two miniature inductors 200 wired in parallel to form a group 215, 225, two such groups 215, 225 being wired in series. As can be understood from TABLE 1 and FIG. 5, such a configured band stop filter 160, 190 has the same curve as a single inductor 240. Specifically, as shown in FIG. 5 by arrow A, the combination parallel and series band stop filter 160, 190 discussed above has the same impedance as a single LC tank 240, as indicated by arrow B. This is because two inductors in parallel would have half of the impedance as a single inductor, but two inductors in serial would double the impedance.

TABLE 1

| | | Rs | | | | |
|---|---|---|---|---|---|---|
| | f0/BW | (Ohms) | QL | L | Cp | Peak Z |
| 3 mil wire 90 | 55.7/(58-54) | 82.7 | 13.6 | 3.2 | 2.5 | 15302 |
| turns tip | | | | uH | pF | ohms |

In one embodiment, as can be understood from FIGS. 3B, 4C and 4D, two miniature inductors 200 wired in parallel may form the only group 215 of inductors 200 for the band stop filter 160, 190. In another embodiment, as can be understood from FIGS. 3B, 4A and 4B, four miniature inductors 200 wired in parallel may form the only group 215 of inductors 200 for the band stop filter 160, 190. If the miniature inductors 200 are selected correctly with respect to peak impedance, SRF and Q, then such parallel only combinations of miniature inductors 200 may be employed to achieve the same impedance at frequency response as a single inductor while achieving circuit redundancy and reduced component heating.

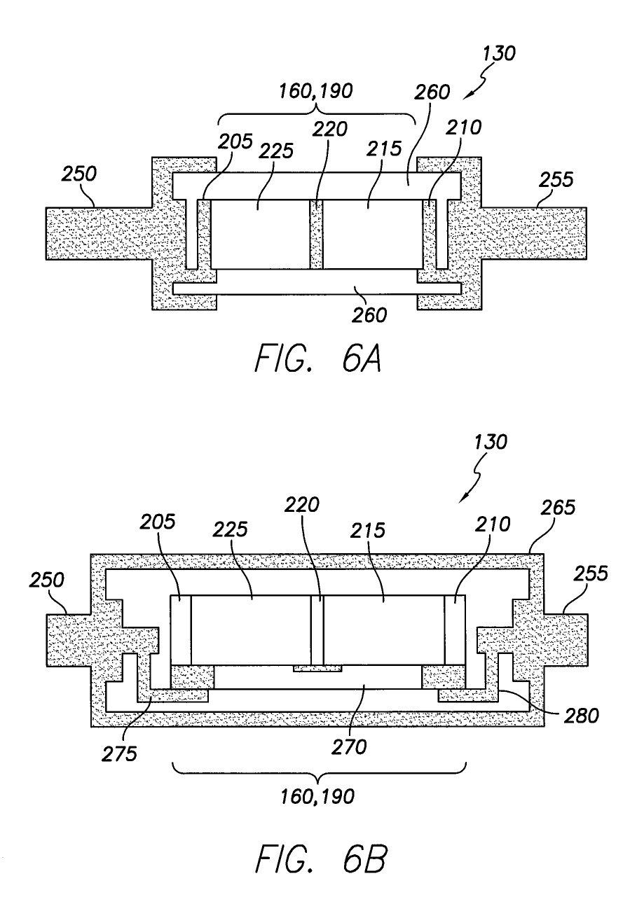

As can be understood from FIGS. 6A and 6B, which are diagrammatic side views of different embodiments of a band stop filter assembly 130 that may be employed in a circuit leading to a tip electrode 75, the band stop filter assembly 130 may or may not employ a hermetic seal. For example, in one embodiment as indicated in FIG. 6A, which does not employ a hermetic seal, the common proximal electrical contact 205 is electrically coupled via a proximal metal member 250 (e.g., the coupler 125 of FIG. 2) to the electrical circuit 85 leading from the band stop filter 160, 190 to the corresponding electrical contact 55 of the lead connector end 35 (see FIG. 1). The common distal electrical contact 210 is electrically coupled via a distal metal member 255 (e.g., the helix base 170 of FIG. 2) to the electrical circuit leading from the band stop filter 160, 190 to the helical anchor electrode 75 (see FIG. 2). The distal group 215 of miniature inductors 200 wired in parallel, as described above with respect to FIGS. 4A-4D, is located between and electrically coupled to the common distal electrical contact 210 and the common intermediate electrical contact 220. The proximal group 225 of miniature inductors 200 wired in parallel, as described above with respect to FIGS. 4A-4D, is located between and electrically coupled to the common proximal electrical contact 205 and the common intermediate electrical contact 220. The distal and proximal inductor groups 215, 225 end up being groups 215, 225 of parallel wired inductors 200, as discussed above with respect to FIGS. 4A-4D, that are wired in series via the common intermediate electrical contact 220, as described above with respect to FIG. 3A.

As shown in FIG. 6A, the inductor groups 215, 225 and common electrical contacts 205, 210, 220 are embedded inside a housing 260 formed of a polymer material, such as, for example, PEEK, and sealed with Med A. The proximal and distal metal members 250, 255 are respectively located at the proximal and distal ends of the polymer housing 260. Thus, the proximal and distal metal members 250, 255, which are respectively in electrical contact with the proximal and distal common electrical contacts 205, 210, can be used to couple the band stop filter 160, 190 to the rest of the electrical circuit leading from the lead connector end 35 to the corresponding electrode 75, 80. Also, the metal members 250, 255 can hold the polymer housing 260. The housing 260 and overall configuration of the band stop filter assembly 130 of FIG. 6A eliminates the need for a hermetic seal.

In one embodiment as indicated in FIG. 6B, the band stop filter assembly 130 does employ a hermetic seal 265 and a printed circuit (PC) board 270 can be employed to support the components of the band stop filter 160, 190 within the hermetic seal 265 As shown in FIG. 6B, the PC board 270 includes proximal and distal metal portions 275, 280. Proximal and distal metal members 250, 255 respectively extend through the proximal and distal ends of the hermetic seal 265 and are respectively electrically coupled to the proximal and distal metal portions 275, 280. Thus, the common proximal electrical contact 205 is electrically coupled via the proximal metal portion 275 and the proximal metal member 250 (e.g., the coupler 125 of FIG. 2) to the electrical circuit leading from the band stop filter 160, 190 to the corresponding electrical contact 55 of the lead connector end 35 (see FIG. 1). Also, the common distal electrical contact 210 is electrically coupled via the distal metal portion 280 and distal metal member 255 (e.g., the helix base 170 of FIG. 2) to the electrical circuit leading from the band stop filter 160, 190 to the helical anchor electrode 75 (see FIG. 2).

As can be understood from FIG. 6B, the distal group 215 of miniature inductors 200 wired in parallel, as described above with respect to FIGS. 4A-4D, is located between and electrically coupled to the common distal electrical contact 210 and the common intermediate electrical contact 220. The proximal group 225 of miniature inductors 200 wired in parallel, as described above with respect to FIGS. 4A-4D, is located between and electrically coupled to the common proximal electrical contact 205 and the common intermediate electrical contact 220. The distal and proximal inductor groups 215, 225 end up being groups 215, 225 of parallel wired inductors 200, as discussed above with respect to FIGS. 4A-4D, that are wired in series via the common intermediate electrical contact 220, as described above with respect to FIG. 3A.

As shown in FIG. 6B, the inductor groups 215, 225, common electrical contacts 205, 210, 220, PC board 270 and metal portions 275, 280 are embedded inside the hermetic seal 265. The proximal and distal metal members 250, 255 are respectively located at the proximal and distal ends of the hermetic seal 365. Thus, the proximal and distal metal members 250, 255, which are respectively in electrical contact with the proximal and distal metal portions 275, 280 and, as a result, the common electrical contacts 205, 210, can be used to couple the band stop filter 160, 190 to the rest of the electrical circuit leading from the lead connector end 35 to the corresponding electrode 75, 80.

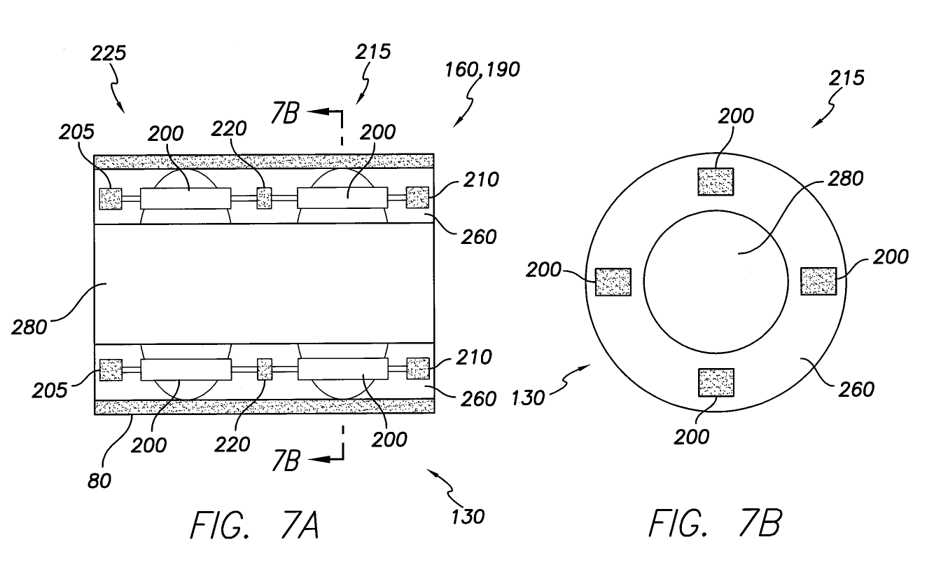

An embodiment of the band stop filter assembly 130 may be configured for use in a circuit leading to a ring electrode 80. For a discussion of such an embodiment, reference is made to FIGS. 7A-7B. FIG. 7A is a diagrammatic side view of the embodiment of a band stop filter assembly 130, and FIG. 7B is a transverse cross section of the band stop filter assembly 130 as taken along section line 7B-7B of FIG. 7A.

In one embodiment, the band stop filter assembly 130 is located proximal the proximal edge of the ring electrode 80 or distal the distal edge of the ring electrode 80. In other embodiments, as shown in FIG. 7A, the band stop filter 130 is located radially inward of the ring electrode 80. In such an embodiment, the common proximal electrical contact 205, which may be in the form of a ring or donut, is electrically coupled to the electrical circuit 90 leading from the band stop filter 160, 190 to the corresponding electrical contact 60 of the lead connector end 35 (see FIG. 1). The common distal electrical contact 210, which may be in the form of a ring or donut, is electrically coupled to the ring electrode 80. In some embodiments, the electrical coupling between the common distal electrical contact 210 and the ring electrode 80 is via direct physical contact.

The distal group 215 of miniature inductors 200 wired in parallel, as described above with respect to FIGS. 4A-4D, is located between and electrically coupled to the common distal electrical contact 210 and the common intermediate electrical contact 220, which may be in the form of a ring or donut. The proximal group 225 of miniature inductors 200 wired in parallel, as described above with respect to FIGS. 4A-4D, is located between and electrically coupled to the common proximal electrical contact 205 and the common intermediate electrical contact 220. The distal and proximal inductor groups 215, 225 end up being groups 215, 225 of parallel wired inductors 200, as discussed above with respect to FIGS. 4A-4D, that are wired in series via the common intermediate electrical contact 220, as described above with respect to FIG. 3A.

In one embodiment as shown in FIGS. 7A and 7B, the band stop filter assembly 130 has a hollow cylinder shape, defining a cylindrical void 280 that extends through the band stop filter assembly 130 to allow components of the lead 10 radially inward of the ring electrode 80 to extend through the band stop filter assembly 130 (see FIG. 2). Depending on the embodiment, the inductor groups 215, 225 and common electrical contacts 205, 210, 220 are embedded inside a housing 260 formed of a polymer material, such as, for example, PEEK, and sealed with Med A. Alternatively, the inductor groups 215, 225 and common electrical contacts 205, 210, 220 are enclosed in a hermetic seal.

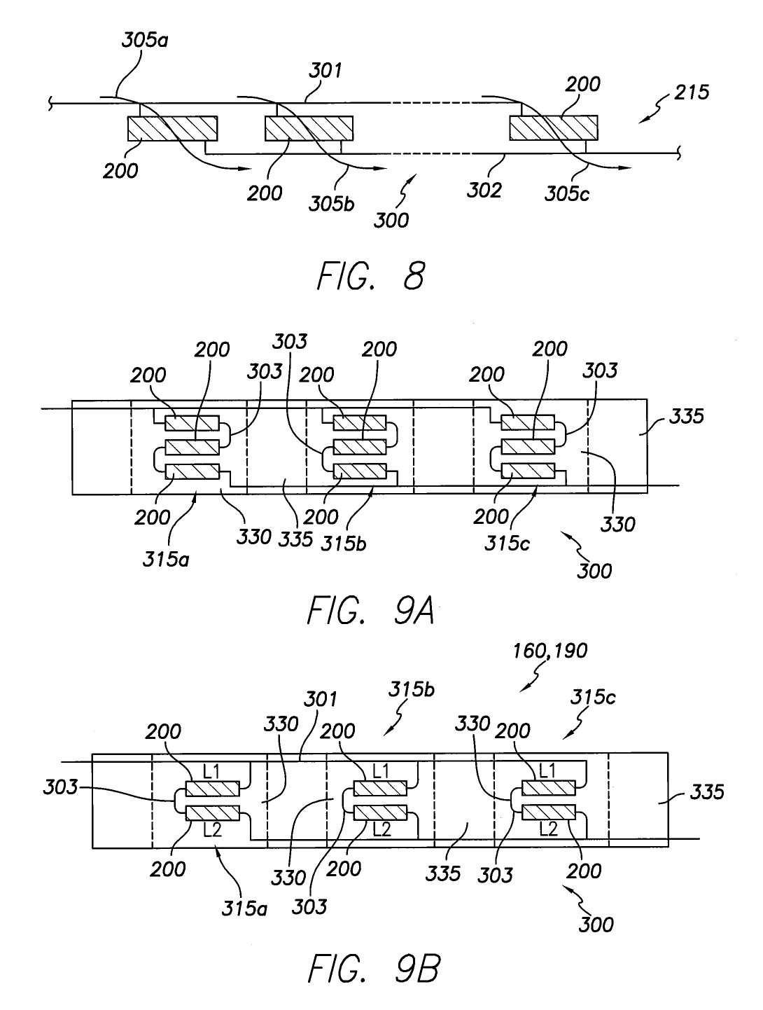

As can be understood from FIG. 8, which is a diagrammatic depiction if a micro-inductor circuit 300, the circuit 300 can have an electrical path 301 into a group 215 of parallel wired micro-inductors 200 and an electrical path 302 out of the group 215 of parallel wired mirco-inductors 200. Connecting the two, three or more micro-inductors 200 in parallel provides two, three or more redundant electrical paths 305a, 305b, 305c as an electrical safety measure for protection against the failure of the micro wire used in a micro-inductor 200.

As shown in FIGS. 9A and 9B, which are plan views of other micro-inductor circuits 300, the micro-inductors 200 can be connected both in series and in parallel. Specifically and unlike the embodiments discussed above, a first group 315a of micro-inductors 200 is wired in series via intermediate conductors 303. This group 315a of micro-inductors 200 is wired between the two electrical paths 301, 302. A second group 315b of micro-inductors 200, a third group 315c of micro-inductors 200, and so forth are each wired in series in a manner similar to that of the first group 315a. Each of the groups 315a, 315b, 315c are wired in parallel between the two electrical paths 301, 302.

As can be understood from FIGS. 9A and 9B, by connecting in series two, three or more micro-inductors in each group 315a, 315b, 315c and then connecting the groups 315a, 315b, 315c in parallel, the value of the overall inductance is increased, heat dissipation is improved, and a small physical size of the band stop filter 160, 190 is achieved. The serially connected inductors 200 may be embedded in an inflexible substrate material 330 to create a serial inductor unit 315a, 315b, 315c. Each of these inflexible substrate mounted serial inductor units 315a, 315b, 315c may be mounted on another substrate 335, which also may be inflexible or, as discussed below, flexible. Two, three or more serial inductor units 315a, 315b, 315c can be combined in parallel to provide two or more redundant electrical path as a safety measure.

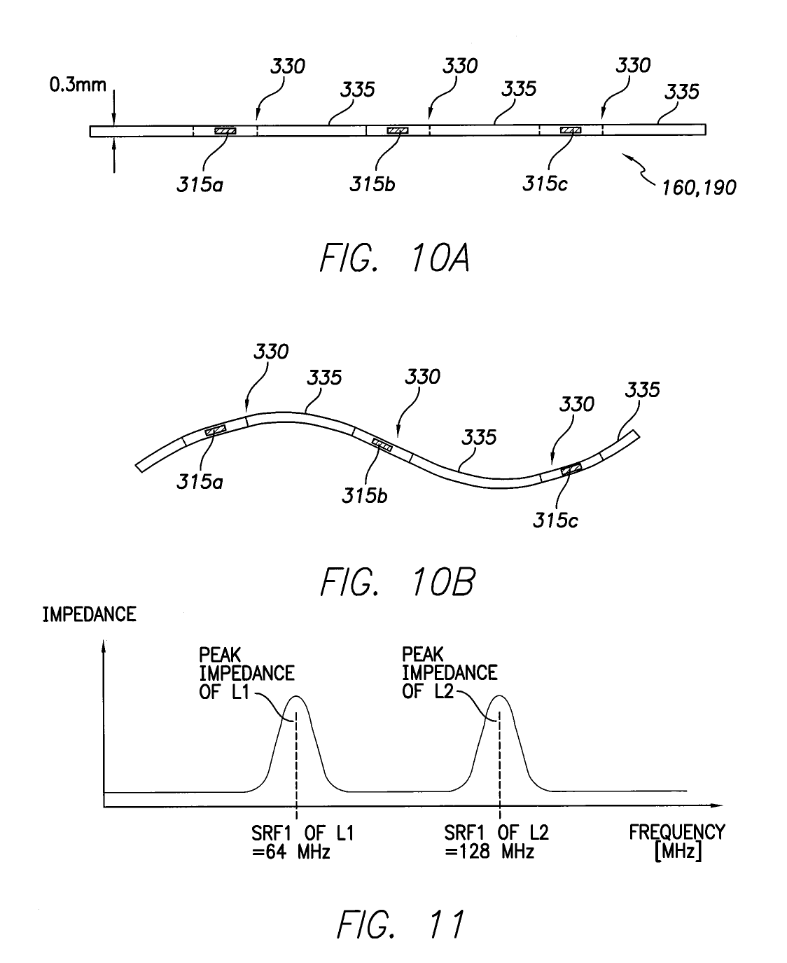

As can be understood from FIGS. 10A and 10B, which are side views of a flexible substrate of the embodiment depicted in FIG. 9A not flexed and flexed, respectively, the inflexible substrates 330 embedding the groups 315a, 315b, 315c of micro-inductors 200 may be interconnected with a flexible substrate 335 that would allow bending of the parallel combination or, in other words, the band stop filter 160, 190. The flexible substrate 335 may be made of one or more materials. In the case of a substrate made of the same material, the flexibility of a given section of the substrate may be controlled by varying its thickness. Thicker substrate sections will have less flexibility and vice versa. Both the thickness and the material of the electrical conductor wires are selected such that the conductor wires can withstand long-term mechanical stresses and fatigue. In one embodiment, the groups 315a, 315b, 315c of micro-inductors 200 may be spaced along the flexible substrate 335 at a spacing of approximately one quarter or less of a wavelength.

As can be understood from FIG. 11, which is a graph depicting peak impedances for band stop filters 160, 190 disclosed herein with respect to FIG. 9B, by combining two, three or more micro-inductors as discussed above, wherein each micro-inductor 200 has a different self-resonant-frequency (SRF), a total impedance may be provided with peak impedances at each of the SRF frequencies. For example, cascading the inductor L1 having an SRF1=64 MHz with the inductor L2 having an SRF2=128 MHz will create a single attenuator with peak impedances at both 64 MHz and 128 MHz. This configuration may be helpful in attenuating RF currents at different frequencies and therefore allowing the use of a single solution for the creation of an MRI lead that is compatible with both 1.5T MRI that employs 64 MHz and 3T MRI systems that employs 128 MHz.

The band stop filters 160, 190 disclosed herein are advantageous for a number of reasons. For example, such band stop filters 160, 190 can fit into the available in the lead header of 7 Fr or 6 Fr leads for both tip and ring electrodes. Such band stop filters 160, 190 offer increased reliability by using multiple electrical connections of inductors 200 instead of having a single failure point. For example, if one of inductors 200 fails, the lead 10 can continue to perform for normal pacing/sensing and even better RF heating reduction in an MRI. Such band stop filters provide improved control of inductor or component heating by distributing the energy among the inductors. Such band stop filters allow early detection of inductor failure by detecting the change in DCR of the package.

In one embodiment, as indicated in FIG. 2, the inductor packages 160, 190 described herein may be located near the distal end of the lead. In other embodiments, the inductor packages 160, 190 described herein may be located at the proximal end of the lead (e.g., near the lead connector end) or at other locations along the lead.

Although the present invention has been described with reference to illustrated embodiments, persons skilled in the art will recognize that changes may be made in form and detail without departing from the spirit and scope of the invention. Indeed, in other embodiments, one or more additional capacitive elements may be coupled to the lead. Additionally, capacitive elements may be implemented with different filtering techniques. For example, although not described herein, a capacitive element may be used in conjunction with a dual tank filter or other filter. Accordingly, the specific embodiments described herein should be understood as examples and not limiting the scope of the disclosure.

What is claimed is:

1. An implantable medical lead comprising:
a first electrode near a distal portion of the lead; and
a first electrical circuit extending through the lead to the first electrode, the first electrical circuit comprising at least one conductor and a first band stop filter coupled between the conductor and the electrode, the first band stop filter comprising a first group of inductors in parallel and a second group of inductors in parallel, the first group in series with the second group.

2. The lead of claim 1, wherein the first group of inductors includes a self resonant L.

3. The lead of claim 1, wherein the first group of inductors includes a self resonant tank LC.

4. The lead of claim 1, wherein the first group of inductors includes a miniature self resonant L or miniature self resonant tank LC.

5. The lead of claim 1, wherein the first group of inductors includes a self resonant L including DFT wire.

6. The lead of claim 5, wherein the DFT wire includes ETFE outer layer.

7. The lead of claim 1, wherein the first group of inductors includes an integrated circuit of L and C components.

8. The lead of claim 1, wherein the first electrode includes a tip electrode.

9. The lead of claim 8, wherein the tip electrode includes a helical anchor.

10. The lead of claim 1, wherein the first band stop filter is embedded in a polymer material.

11. The lead of claim 10, wherein the polymer material includes PEBAX.

12. The lead of claim 1, wherein the first band stop filter is enclosed in a hermetic seal.

13. The lead of claim 1, wherein the band stop filter further includes a PC board supporting the first and second groups of inductors.

14. The lead of claim 1, further comprising a second electrode near the distal portion of the lead and a second electrical circuit extending through the lead to the second electrode and comprising a second band stop filter comprising a third group of inductors in parallel and a fourth group of inductors in parallel, the third group in series with the fourth group.

15. The lead of claim 14, wherein the first electrode is distal the second electrode.

16. The lead of claim 14, wherein the first electrode includes a tip electrode and the second electrode includes a ring electrode.

17. The lead of claim 14, wherein the second electrode includes a ring electrode and the second band stop filter is located radially inward of the second ring electrode.

18. The lead of claim 1, wherein the first group of inductors is supported by a first inflexible substrate and the second group of inductors is supported by a second inflexible substrate, the first and second inflexible substrates coupled together via a flexible substrate.

19. An implantable medical lead comprising:
a first electrode near a distal portion of the lead; and
a first electrical circuit extending through the lead to the first electrode, the first electrical circuit comprising at least one conductor and a first band stop filter coupled between the conductor and the electrode, the first band stop filter comprising a first group of inductors in series and a second group of inductors in series, the first group in parallel with the second group.

20. The lead of claim 19, wherein the first group of inductors includes at least one of a self resonant L, a self resonant tank LC or an integrated circuit of L and C components.

21. The lead of claim 19, further comprising a second electrode near the distal portion of the lead and a second electrical circuit extending through the lead to the second electrode and comprising a second band stop filter comprising a third group of inductors in series and a fourth group of inductors in series, the third group in parallel with the fourth group.

22. The lead of claim 20, wherein the first electrode includes a tip electrode and the second electrode includes a ring electrode.

23. The lead of claim 19, wherein the first group of inductors is supported by a first inflexible substrate and the second group of inductors is supported by a second inflexible substrate, the first and second inflexible substrates coupled together via a flexible substrate.
